(19) United States
(12) Patent Application Publication
(10) Pub. No.: US 2003/0158137 A1
(43) Pub. Date: Aug. 21, 2003

(54) Gene directed enzyme prodrug theraphy (gdept) for cell ablation

(76) Inventor: Donald Davies, Buckinghamshire (GB)

Correspondence Address: COZEN O'CONNOR, P.C., 1900 MARKET STREET, PHILADELPHIA, PA, 19103-3508, US

(21) Appl. No.: 10/258,760

(22) PCT Filed: Apr. 25, 2001
(86) PCT No.: PCT/GB01/01828

(30) Foreign Application Priority Data
Apr. 26, 2000 (GB) 0010105.5

(51) Int. Cl. A61K 48/00

(52) U.S. Cl. 514/44; 424/93.2

(57) ABSTRACT

The invention relates to a method of gene directed enzyme pro-drug therapy (GDEPT) in the ablation of cells wherein said cells are not cancerous cells.

The invention relates to the use of gene directed enzyme prodrug therapy (GDEPT) in the ablation of cells, wherein said cells are not cancerous cells, the removal of which has therapeutic benefit.

There are many disease conditions, other than the ablation of cancer cells, which would benefit from the specific removal of cell types. For example, and not by way of limitation, this includes the ablation of epidermal cells (eg keratinocytes) in psoriaisis, the ablation of cells infected with viruses or the ablation of endothelial cells which form blood vessels which vascularise tumours.

Enzyme prodrug therapy (EPT) relates to the use of enzymes which convert a relatively inert or non-toxic drug, a prodrug, into a cytotoxic agent when the enzyme metabolises the prodrug, see Connors Gene Therapy, 2, p702, (1995). EPT exploits endogenous enzymes to activate prodrugs. Tumour cells in particular contain many prodrug activating enzymes (eg cytochrome P450) in sufficient amounts to convert a prodrug into its active derivative thereby damaging the tumour cell sufficiently to render it unviable. An alternative to EPT is so called Gene Directed Enzyme Prodrug Therapy, GDEPT. This has been developed as an alternative means to activate a prodrug in a cell/tissue specific manner. The method involves the transfection of a vector encoding a prodrug enzyme controlled by a promoter sequence which has restricted expression in so far as the promoter has elevated activity in a specific cell type. This approach allows the use of non-human prodrug enzymes or genetically engineered prodrug enzymes which have increased activity. This has the added advantage that an endogenous prodrug enzyme equivalent does not exist in the animal to be treated therefore the production of the active drug, in for example, the liver, does not occur.

An example of this is the thymidine kinase enzyme of herpes simplex virus. This enzyme phosphorylates the prodrug gancyclovir to produce a nucleotide analogue which is an effective inhibitor of DNA synthesis, (Moolten, Cancer Res. 46, 5276). The activated drug is S-phase specific (that period of the cell-cycle during which DNA synthesis occurs) and kills cells which are actively dividing. This is a disadvantage in so far as there is a restricted window during which the drug is active and non-tumour cells, which are proliferating, are also targetted resulting in undesirable side effects.

In known GDEPT techniques, difficulty has been encountered in achieving as high a degree of selectivity as is desirable, (ie in destroying diseased cells whilst limiting the damage to normal cells). This is at least partly due to the fact that normal cells may come under attack from cytotoxic agents which have been formed in the diseased cells but have found their way out of those cells, for example when the cells break down under the cytotoxic action of the drug. In addition, as mentioned above, some agents are selective for particular cell-cycle phases ( eg G1, S, G2 or mitosis). It is desirable to provide agents that are not so restricted in their effects and can kill cells irrespective of the cell-cycle stage.

WO00/40271 and WO00/40272 disclose the use of acetaminophen as a prodrug to ablate tumour cells. The present application relates to the use of acetaminophen as a prodrug to ablate cells other than tumour cells. For example, epidermal cells in psoriasis, endothelial cells which form blood vessels which vascularise tumours and cells which have become infected with viruses.

Skin is a highly complex organ covering the external surface of the body. Skin functions, amongst other things, to prevent water loss from the body and to act as a protective barrier against the action of physical, chemical or infectious agents.

Skin is composed of two layers. The outer layer, which is comparatively thin, is called the epidermis. It is several cells thick and has an external layer of dead cells that are constantly shed from the surface and replaced from below by a basal layer of cells, called the stratum germinativum. The epidermis is composed predominantly of keratinocytes which make up over 95% of the cell population, the rest include dendritic cells such as Langerhans and pigmented cells called melanocytes. It is essentially cellular and non vascular, there being relatively little extra cellular matrix except for the layer of collagen and other proteins beneath the basal layer of keratinocytes.

The inner layer of the skin is called the dermis and is composed of a network of collagenous extracellular material, elastic fibres, blood vessels and nerves. Contained within it are hair follicles and associated sebaceous glands, collectively known as the pilosebaceous unit. The interface between the epidermis and dermis is extremely irregular and consists of a succession of papillae, or finger like projections. Skin is therefore a highly complex organ the cells of which are periodically replaced.

Skin is susceptible to both physical damage and a number of debilitating although not life threatening diseases. One example of such a disease is psoriasis. Psoriasis is a generic term to cover a range of diseases charactersised by abnormal proliferation of skin cells. The disease covers the following list which is not exhaustive but merely illustrative: nail psoriasis; scalp psoriasis; plaque psoriasis; pustular psoriasis; guttate psoriasis; inverse psoriasis; erythrodermic psoriasis; psoriatic arthritis.

Psoriasis is one of the most frequent skin diseases, affecting 1-3% of the Caucasian population world wide. The disease is characterised by alterations in a variety of different cell types. These include epidermal keratinocytes which are characterised by hyperproliferation and an altered differentiation which is indicated by focal parakeratosis and aberrant expression of keratinocyte genes encoding hyperproliferation-associated keratin pair 6/16, involucrin, fillagrin, and integrin adhesion molecules (eg VLA-3, 5, 6).

Current methods to control psoriatic conditions include the use of topical applications of coal tar which reduce itching and scaling of skin. However, coal tar sensitises skin to ultraviolet thereby rendering individuals susceptible to sunburn. Treatment with coal tar can also result in photosensitivity. An alternative to the use of coal tar is topical steroids. Although effective, steroid treatment can result in thinning of skin. Also if steriods are used longterm the body can become resistant thereby rendering the treatment ineffective. Other pharmaceutical treatments include the topical application of anthralin, vitamin D3 and retinoid treatment. Oral medications are also available to those with severe forms of the disease which do not respond well to topical treatments. These include methotrexate, cyclosporins, Tegison. Each of these medications have serious side effects which include liver and lung damage (methotrexate) immunosuppression (cyclosporins) and rashes, hair loss and hepatitis (Tegison). Also many of these drugs are incompatible with pregnancy and therefore should be avoided by women of childbearing age.

Clearly there is a need for alternative treatments which do not have the above identified disadvantages.

When normal keratinocytes are cultured, they assume a hyperproliferative state that is similar to psoriasis in vivo and has been labelled the pseudo-psoriatic phenotype. This provides an excellent model of testing various therapeutic treatments in vitro before animal model experiments are undertaken. Moreover, there exists an animal model for psoriasis which allows the testing of various therapies with respect to the treatment of this condition, see U.S. Pat. No. 5,945,576, the contents of which are incorporated by reference.

In treatment of skin disorders by GDEPT the objective is to create an anti-psoriatic drug in situ within the psoriatic cell while creating little or none in normal cells. This is typically achieved by administration to the patient of a vector containing a gene for an enzyme which can convert a relatively non-toxic substance (commonly referred to as a prodrug) into a cytotoxic agent. The vector also contains a promoter, (ie a DNA sequence controlling the transcription of a gene), this promoter being responsive to a regulatory protein found solely in the psoriatic cells or to a greater extent in the psoriatic cells than in normal cells. This can be simply done by topically administering the vector, which includes an cell specific promoter controlling a prodrug activating enzyme, to a psoriatic plaque thereby localising the vector to those cells undergoing hyperproliferation.

Angiogenesis, the development of new blood vessels from an existing vascular bed, is a complex multistep process that involves the degradation of components of the extracellular matrix and then the migration, proliferation and differentiation of endothelial cells to form tubules and eventually new vessels. Angiogenesis is important in normal physiological processes including, by example and not by way of limitation, embryo implantation; embryogenesis and development; and wound healing. Angiogenesis is also involved in pathological conditions such as tumour cell growth; non-cancerous conditions such as neovascular glaucoma; inflammation; diabetic nephropathy; retinopathy; rheumatoid arthritis; inflammatory bowel diseases (eg Crohn's disease, ulcerative colitis); and psoriasis.

The vascular endothelium is normally quiescent. However upon activation endothelial cells proliferate and migrate to form microtubules which will ultimately form a capillary bed to supply blood to developing tissues and, of course, a growing tumour. A number of growth factors have been identified which promote/activate endothelial cells to undergo angiogenesis. These include, by example and not by way of limitation; vascular endothelial growth factor (VEGF); transforming growth factor (TGFb); acidic and basic fibroblast growth factor (aFGF and bFGF); and platelet derived growth factor (PDGF), Folkman, Nature Medicine, 1: 27-31, 1995; Leek et al J. Leuk. Biol., 56: 423-35, 1994.

VEGF is a an endothelial cell-specific growth factor which has a very specific site of action, namely the promotion of endothelial cell proliferation, migration and differentiation. VEGF is a dimeric complex comprising two identical 23 kD polypeptides. The monomeric form of VEGF can exist as four distinct polypeptides of different molecular weight, each being derived from an alternatively spliced mRNA. Of the four monomeric forms, two exist as membrane bound VEGF and two are soluble. VEGF is expressed by a wide variety of cell/tissue types including embryonal tissues; proliferating keratinocytes; macrophages; tumour cells. Studies (2) have shown VEGF is highly expressed in many tumour cell-lines including glioma and AIDS associated Karposi's sarcoma. VEGF activity is mediated through VEGF specific receptors expressed by endothelial cells and tumour cells. Indeed the VEGF receptor is up-regulated in endothelial cells which infiltrate tumours thereby promoting tumour cell growth.

Endothelial cell turnover in healthy adult organisms is very low. The maintenance of quiescent endothelial cells is thought to be due to the presence of endogenous negative regulators of angiogenesis. In activated endothelial cells, positive activators, for example the above identified growth factors, predominate. It has therefore been hypothesised that a “molecular switch” exists which modulates the induction of positive activator(s) and the inactivation of negative regulator(s). The role of the VEGF family of growth factors in tumour angiogenesis has been established, Carmeliet et al Nature, 380: 453-39; Ferrara et al Endocrine Rev. 18: 4-25. The endothelial function of VEGF family members is transduced via transmembrane tyrosine kinase receptors (eg VEGFR-1, VEGFR-2, VEGFR-3), see Mustonen et al. J Cell Biol. 129: 895-898. These receptors show upregulation during development and certain pathological conditions such as tumour growth, Dvorak et al. Amer. J.Pathol., 146:1029-1039; Ferrara et al. Endocrine Rev., 18: 4-25. All of these are incorporated by reference.

Other examples of genes expressed in activated endothelial cells include, by example and not by way of limitation, are brain specific, endothelial glucose-1-transporter (Murakami et al J.Biol Chem., 267, 9300); endoglin (Ge et al Gene 138, 201); B61 receptor (Bartley et al Nature 368, 558); endothelin B (Benetti et al J Clinical Invest. 91, 1149); mannose-6-phosphate (Ludwig et al Gene 142, 311); IL-1 α, IL-1 β(Hangen et al Mol Carcinog. 2, 68); IL-1 receptor (Ye et al PNAS USA 90, 2295). Each of the above references describe promoter sequences involved in regulating the endothelial specific expression of the identified genes. Each of which are incorporated by reference.

Moreover, vectors used to transform endothelial cells which include the above identified promoters are well known in the art and are described in, for example U.S. Pat. No. 5,830,880, the content of which is incorporated by reference.

Viruses cause amongst the most debilitating and life threatening diseases known to mankind. Examples of viruses which cause such diseases include, by example and not by way of limitation: retroviruses eg Human Inmmunodeficiency Virus (HIV1 & 2); Human T Cell Leukamia Virus (HTLV 1 & 2); Ebola virus; papilloma virus; papovavirus; rhinovirus; poliovirus; herpesvirus; adenovirus; Epstein barr virus; and influenza virus.

A virus exploits the replication and transcription machinery of the cell infected. When a virus infects a cell a number of intracellular events occur in response to infectivity which results in the induction of specific gene expression by the genes encoded by the cells genome. An example of this is the interferon β gene, see Parekh and Maniatis, (1999), Molecular Cell, 3, 125-129. The interferon β gene promoter is controlled by a number of transcriptional activators which become active when a virus infects. For example, when Sendai virus infects the transcriptional activators NF-κB, ATF-2C-jun, IRE-3, IRE-7, HMG(Y) and the co-activators p300 and CBP assemble at the interferon β promoter to activate transcription.

U.S. Pat. No. 6,043,351, the content of which is incorporated by reference, discloses a family of genes which are transcriptionally activated when Epstein Barr virus (EBV) infects. These genes, collectively referred to as Epstein Barr Inducible (EBI), EBI 1, EBI 2, EBI 3. EBV causes infectious mononucleosis, a benign proliferation of infected B-lymphocytes (Henle et al PNAS USA 59(1):94-101, 1968) and can cause acute and rapidly progressive B lymphoproliferative disease in severely immunosuppressed patient.

In its broadest aspect the invention provides the means to specifically ablate cells, the removal of which has beneficial therapeutic effects comprising:

(i) administering to a mammal an effective amount of at least one vector capable of transfecting a cell characterised in that said vector includes at least one P450 gene, or an effective part thereof, the expression of which is controlled by a promoter sequence, or the effective part thereof, which shows substantially cell specific expression; and

(ii) administering a therapeutically effective amount of at least acetaminophen, or a structurally related variant thereof.

Acetaminophen is a widely used mild pain reliever and antipyretic. However, it is a potentially dangerous drug in that an overdose of it can cause serious, even fatal, damage to the liver. This is due to the fact that liver cells express a gene for a P450 enzyme, specifically CYP1A2. This enzyme can convert acetaminophen into a metabolite, N-acetylbenzoquinoneimine (NABQI), which is highly cytotoxic. Liver cells also express other P450 genes, CYP 2E1 and CYP 3A4. These enzymes are found in higher levels in the liver but are less efficient than CYP1A2 at converting acetaminophen to NABQI. For standard dosages of acetaminophen, the toxicity of NABQI is countered in the liver by conversion of NABQI into a non-toxic substance by reaction with glutathione, a normal component of human cells. The supply of glutathione is however insufficient to deal with the large amounts of NABQI formed in liver cells after an overdose of acetaminophen and the cells are therefore then damaged or destroyed.

When acetaminophen constitutes the prodrug in GDEPT, the vector administered may contain a gene for a p450 enzyme, preferably CYP1A2, and in this case, the cytotoxic agent formed in the transfected cells is NABQI. In contrast to other cytotoxic agents, NABQI causes little or no systemic toxicity.

Selective expression of the gene for the enzyme CYP1A2 in cells could be effected by administration of a vector containing that gene in association with a promoter which is responsive to a regulatory protein found only in said cells. The enzyme CYP1A2, created as a result of the entry into cells of that vector, would then convert acetaminophen into NABQI in the psoriatic cells and damage or destroy them. As in conventional GDEPT using prodrugs other than acetaminophen, selectivity between diseased/abnormal cells and normal cells would be achieved because entry of the vector into normal cells would not result in expression of P450. In addition it would not be necessary to effect transfection of all cells since the production of NABQI would have a so-called “bystander effect” on surrounding, non-transfected cells, see Freeman et al Cancer Res. 53, 5274, (1993).

In a further preferred method of the invention said mammal is human.

In yet a further preferred method of the invention said vector is an expression vector conventionally adapted for eukaryotic expression.

Typically said adaptation includes, by example and not by way of limitation, the provision of transcription control sequences (promoter sequences) which mediate cell/tissue specific expression. These promoter sequences may be cell/tissue specific, inducible or constitutive.

Promoter is an art recognised term and, for the sake of clarity, includes the following features which are provided by example only, and not by way of limitation. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors(please see Eukaryotic Transcription Factors, by David S Latchman, Academic Press Ltd, San Diego) is responsive to a number of environmental cues which include, by example and not by way of limitation, intermediary metabolites (eg glucose, lipids) or environmental effectors (eg light, heat,).

Promoter elements also include so called TATA box and RNA polymerase initiation site (RIS) sequences which function to select a site of transcription initiation. These sequences also bind polypeptides which function, inter alia, to facilitate transcription initiation selection by RNA polymerase.

Adaptations also include the provision of selectable markers and autonomous replication sequences which both facilitate the maintenance of said vector in either the eukaryotic cell or prokaryotic cell.

In addition adaptations which facilitate the expression of vector encoded genes include the provision of transcription termination/polyadenylation sequences. This also includes the provision of internal ribosome entry sites (IRES) which function to maximise expression of vector encoded genes arranged in bicistronic or multi-cistronic expression cassettes.

These adaptations are well known in the art. There is a significant amount of published literature with respect to expression vector construction. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK.

In yet a further preferred method of the invention said vector is a viral based vector. Ideally said viral vector is selected from the following: adenovirus; retrovirus; adeno associated virus; herpesvirus; lentivirus; baculovirus.

Viral based vectors according to the invention may also include hybrid viral vectors which include advantageous features of selected viruses which facilitate, for example and not by way of limitation, viral infectivity, replication or expression of genes carried by said hybrid vector.

Conventional methods to introduce DNA into cells are well known in the art and typically involve the use of chemical reagents, cationic lipids or physical methods. Chemical methods which facilitate the uptake of DNA by cells include the use of DEAE-Dextran (Vaheri and Pagano Science 175: p434). DEAE-dextran is a negatively charged cation which associates and introduces the DNA into cells but which can result in loss of cell viability. Calcium phosphate is also a commonly used chemical agent which when co-precipitated with DNA introduces the DNA into cells (Graham et al Virology (1973) 52: p456).

The use of cationic lipids (eg liposomes (Felgner (1987) Proc.Natl.Acad.Sci USA, 84:p7413) has become a common method since it does not have the degree of toxicity shown by the above described chemical methods. The cationic head of the lipid associates with the negatively charged nucleic acid backbone of the DNA to be introduced. The lipid/DNA complex associates with the cell membrane and fuses with the cell to introduce the associated DNA into the cell. Liposome mediated DNA transfer has several advantages over existing methods. For example, cells which are recalcitrant to traditional chemical methods are more easily transfected using liposome mediated transfer.

More recently still, physical methods to introduce DNA have become effective means to reproducibly transfect cells. Direct microinjection is one such method which can deliver DNA directly to the nucleus of a cell (Capeechi (1980) Cell, 22:p479). This allows the analysis of single cell transfectants. So called “biolistic” methods physically shoot DNA into cells and/or organelles using a particle gun (Neumann (1982) EMBO J, 1: p841). Electroporation is arguably the most popular method to transfect DNA. The method involves the use of a high voltage electrical charge to momentarily permeabilise cell membranes making them permeable to macromolecular complexes. However physical methods to introduce DNA do result in considerable loss of cell viability due to intracellular damage. These methods therefore require extensive optimisation and also require expensive equipment.

In a further preferred method of the invention said P450 gene is of mammalian origin; ideally human. More ideally still said P450 gene is human CYP1A2. Alternatively said P450 gene is either human CYP2E1 or CYP3A4.

In yet still a further preferred method of the invention said P450 is of non-human origin. Ideally said P450 gene is derived from a rodent. More ideally still said rodent P450 gene is selected from homologous rodent genes encoding CYP1A2; CYP2E1, or CYP3A4.

The vector used in the present invention is one containing a gene for a p450 enzyme, preferably for CYP1A2, and a promoter which controls expression of that gene and which is responsive to a trans acting factor characteristic of that cell. The gene can be derived from human DNA (Ikeyak et al Molecular Endocrinology (1989), 3: 1399-1408). However, it may be advantageous to use a P450 gene derived from non-human DNA, for example mouse DNA or hamster DNA. The P450 enzyme generated by the mouse gene is relatively unaffected by certain compounds, for example furaphylline, which act as inhibitors of the form of the enzyme CYP1A2 generated by the human gene. Administration of such inhibitors makes it possible to raise the dosage of acetaminophen above the normally safe dosage; an inhibitor such as furaphylline can protect the normal liver cells by inhibiting the form of P450 generated by expression in those cells of the human gene, while having little or no effect on the form of P450 generated by expression of the mouse gene in the transfected cells. The level of NABQI in the normal liver cells is therefore diminished by such inhibitors while the level of NABQI in the transfected cells is relatively unaffected by the inhibitors.

In a preferred method of the invention when said method includes the use of a rodent homologue of CYP1A2, CYP2E1 or CYP3A4 said mammal is additionally administered an amount of an inhibitor of human CYP1A2, CYP2E1 or CYP3A4. Ideally said inhibitor is furaphylline, or variant thereof. It will be apparent that the administration of the inhibitor may be prior to, coincident with or after administration of the vector DNA and/or acetaminophen.

According to a further aspect of the invention there is provided a therapeutic composition comprising the vector used in the method according to the invention.

In a preferred embodiment of the invention said therapeutic composition comprises an excipient, diluent or carrier.

According to a yet further aspect of the invention there is provided a therapy for the treatment of cells comprising:

(i) administering to a mammal an effective amount of at least one vector capable of transfecting a cell characterised in that said vector includes at least one P450 gene, or an effective part thereof, the expression of which is controlled by a promoter sequence, or the effective part thereof, which shows substantially cell specific expression;

(ii) adminstering an effective amount of at least one agent capable of modulating the amount of glutathione in the liver of said mammal; and

(iii) administering a therapeutically effective amount of at least acetaminophen, or a structurally related variant thereof.

It is well known in the art that glutathione detoxifies NABQI. Therefore agents which increase the effective amount of glutathione in liver may allow the use of elevated amounts of acetaminophen to be used to treat diseased/abnormal cells. Agents capable of increasing glutathione in the liver are well known in the art and include, by example and not by way of limitation, methionine, acetylcysteine.

In a further preferred method of the invention acetaminophen and said agent capable of modulating glutathione are provided as a combined preparation for simultaneous, separate or sequential use in the treatment of diseased/abnormal cells.

It will also be apparent to one skilled in the art that acetaminophen can be administered in different forms. For example and not by way of limitation, acetaminophen can be added directly to the therapeutic composition and applied topically. Alternatively acetaminophen can be taken orally, in unit dosage form, to maintain a sufficient systemic concentration to effectively kill transfected cells expressing P450.

In accordance with any previous aspect or embodiment of the invention preferably said cell is a psoriatic cell. Ideally said cell is a keratinocyte.

It will be apparent to one skilled in the art that the majority of cells (>95%) which comprise skin are keratinocytes at various stages of differentiation. Keratinocytes of the basal layer are constantly dividing and daughter cells subsequently move outwards, during which they undergo a period of differentiation and arrest cell division. It is the uncontrolled division of these keratinocytes which result in the formation of psoriatic plaques.

In a preferred method of the invention said promoter sequences are selected from the following: keratin promoters K1; K5; K6; K10; K14; filaggrin; loricrin; involucurin.

Ideally said promoter sequence is keratin K6. It is known that the K6 promoter shows a high level of expression in epidermal cells undergoing hyperproliferation, see U.S. Pat. No. 5,958,764, the content of which is incorporated by reference.

It will be apparent to one skilled in the art that genes which show the requisite expression pattern are readily available. For example, and not by way of limitation, keratin K6, K5 and K14 are described in Woodcock and Mitchell, J. Cell Biol. 95, p580-88 (1982); K1 and K10 are described in Roop et al Proc Natl. Acad.Sci USA, 80, p716-720, (1983) and Schweizer et al, Cell 37, p159-170, (1984). Many of these genes have been cloned and their sequences published, for example, K5, Lersch et al Mol Cell Biol. 8, p486-493, (1988); K14, Marchuk et al Proc.Natl.Acad.Sci.USA, 82, P1609-1613, (1985) and Knapp et al J.Biol Chem. 262, 938-945, (1987); K1, Steinert et al., J.Biol.Chem. 260, p7142-7149, (1985); K10, Kreig et al J.Biol.Chem. 260, p5867-5870, (1985); K6, Tyner et al Proc.Natl Acad.Sci.USA, 82, 4683-4687, (1985); loricrin, Yoneda et al. J.Biol.Chem. 267(25), 18060-18066; each of which is incorporated by reference.

Moreover, promoter sequences defining the 5′ regions responsible for transcription activation are known, for example see, Tomic et al Cell Reg. 1, p965-973 (K5, K6, K10, K14); Greenhalgh et al, Mol. Carcinogenesis, 7, p99-110, (1993). Methods for transfecting epidermal cells with vectors including epidermal specific promoters are also known as are methods relating to the heterologous expression of polypeptides in epidermal cells, see Morgan et al, Science, 237, p1476-1479, (1987); Teumer et al FASEB, 4, p3245-3250, (1990); Sellheyer et al Proc.Natl.Acad.Sci USA, 90, p5237-5241, (1993). Cell culture methods for keratinocytes are also known, see U.S. Pat. No. 5,968,546. Each of which is incorporated by reference.

The administration of the vector according to the invention to the mammal is by techniques established in the art. This particularly includes, by example and not by way of limitation, topical application of the vector in a suitable excipient.

In accordance with any previous aspect or embodiment of the invention said cell is an endothelial cell.

In a preferred method of the invention said endothelial cell is an activated endothelial cell. Ideally said endothelial cell is involved in the vascularisation of tumours.

In a further preferred method of the invention said cell specific promoter is selected from: VEGFR-1; VEGFR-2; VEGFR-3; brain specific, endothelial glucose-1-transporter; endoglin; B61 receptor; endothelin B; mannose-6-phosphate; IL-1α; IL-1β; IL-1 receptor.

In accordance with any previous aspect or embodiment said cell is a virally infected cell.

In a preferred embodiment of the invention said virally infected cell is infected with: Human Immunodeficiency Virus (HIV1 & 2); Human T Cell Leukamia Virus (HTLV 1 & 2); Ebola virus; human papilloma virus(HPV); papovavirus; rhinovirus; poliovirus; herpesvirus; adenovirus; Epstein barr virus; influenza virus.

In a further preferred method of the invention the HPV is selected from the following: HPV-2; HPV-6; HPV-11; HPV-16, HPV-18, HPV-31, HPV-33, HPV-52, HPV-54; HPV-56; HPV-5 and HPV-8. More preferably still the HPV is HPV-16.

In a further preferred embodiment of the invention the virally induced promoter is interferon β.

In yet a further preferred embodiment of the invention said virally induced promoter is selected from the following: EB 1; EB2; EB3.

TABLE 1
Promoter sequences and Genbank accession numbers
Genbank Web Address
http://www.ncbi.nlm.nih.gov/genbank/query_form.html

| Promoter sequence | Accession number |
|---|---|
| K 12 (Krt 1-12) gene and promoter | AF053090 |
| region | |
| K 4 (KRT 4) gene, promoter and partial | AF066051 |
| cds | |
| K14 (KRT 14) gene and promoter region | U11076 |
| K17 romoter region | S81026 |
| K12(Krt 1.12) gene and partial gene | AF011495 |
| promoter | |
| K19 gene and romoter region | AF089865 |
| K14 5' upstream region sequence | X59475 |
| K 6 | US 5,958,764 |
| K 5 promoter | S56203 |
| K 19 (Krt 1-19) gene, promoter region | AF237661 |
| and 5' UTR | |
| Loricrin promoter | S77319 |
| K10 gene, promoter region and partial | AF245658 |
| cds | |
| VEGF gene, promoter region and partial | AF095785 |
| cds | |
| VEGF gene, promoter and partial | AF098331 |
| VEGF-related factor (VRF) gene and | U80601 |
| VEGF gene, partial cds and promoter | U41383 |
| IL-1 alpha gene | X03833 |
| Endothelin-converting enzyme-1 | AJ011770 |
| Beta-R1 gene and romoter sequence | AF113846 |
| IFN-beta promoter | E00218 |
| FBI 1, EBI 2, EBI 3 | US 6,043,351 |

TABLE 2
Cytochrome P450 coding sequence

| | |
|---|---|
| Human cytoebrome P450-1A2 | AF182274 |
| (CYP1A2) mRNA and complete cds | |
| Human cytochrome P450 (CYP1A2) | AH002667 |
| gene | |
| Human cytochrome P450-2E1 (CYP2E1) | AF182276 |
| mRNA and complete cds | |
| Human P450 polypeptide 4 (CYP3A4) | AF280107 |
| Human cytochrome P450 IIIA4 | AF209389 |
| (CYP3A4) gene, exons 1 through 13 and | |
| complete cds | |
| Rattus norvegicus cytochrome P450 3E1 | AF056333 |
| CYP2E1 mRNA and partial cds | |
| Guinea pig CYP1A2 mRNA and | D50457 |
| complete cds | |
| Hamster CYP2E1 mRNA and complete | D17449 |
| cds | |

1. A method to specifically ablate cells, wherein said cells are not cancer cells, comprising:
i) administering to a mammal an effective amount of a vector capable of transfecting a cell wherein the vector includes at least one P450 gene, or an effective part thereof, the expression of which is controlled by a promoter sequence, or the effective part thereof, which shows substantially cell specific expression; and
ii) administering a therapeutically effective amount of acetaminophen, or a structurally related derivative thereof

2. A method according to claim 1 wherein said mammal is human.

3. A method according to claim 1 or 2 wherein the vector is an expression vector adapted for eukaryotic expression.

4. A method according to any of claims 1-3 wherein the vector is a viral based vector.

5. A method according to claim 4 wherein the viral vector is selected from the following: adenovirus; retrovirus; adeno-associated virus; herpes virus; lentivirus; baculovirus.

6. A method according to any of claims 1-5 wherein the P450 gene is of mammalian origin.

7. A method according to claim 6 wherein the P450 gene is of human origin.

8. A method according to claim 7 wherein the P450 gene is selected from the following group CYP1A2;CYP2E1 or CYP3A4.

9. A method according to any of claims 1-6 wherein the P450 gene is of non-human origin.

10. A method according to claim 9 wherein the P450 gene is of rodent origin.

11. A method according to claim 10 wherein the rodent P450 gene is selected from the homologous rodent gene encoding CYP1A2; CYP2E1 or CYP3A4.

12. A method according to any claims 10 or 11 wherein said mammal is additionally administered an amount of an inhibitor of human CYP1A2, CYP2E1 or CYP3A4.

13. A method according to claim 12 wherein the inhibitor is furaphylline, or a structural variant thereof.

14. A method to specifically ablate cells, wherein said cells are not cancer cells, comprising:
i) administering to a mammal an effective amount of a vector capable of transfecting a cell wherein the vector includes at least one P450 gene, or an effective part thereof, the expression of which is controlled by a promoter sequence, or the effective part thereof, which shows substantially cell specific expression;
ii) administering an effective amount of at least one agent capable of modulating the amount of gluthathione in the liver of said mammal; and
iii) administering a therapeutically effective amount of acetaminophen, or a structurally related variant thereof.

15. A method according to any of claims 1-14 wherein the transfected cell is a psoriatic cell.

16. A method according to claim 15 wherein said cell is a psoriatic keratinocyte.

17. A method according to claim 15 or 16 wherein the promoter sequences are selected from the following list of keratin promoters K1; K5; K6: K10: K14; filaggrin; loricrin; involucurin.

18. A method according to claim 17 wherein the promoter sequence is the keratin promoter K6.

19. A method according to any of claims 1-14 wherein said cell is endothelial cell.

20. A method according to claim 19 wherein the endothelial cell is an activated endothelial cell.

21. A method according to claim 19 or 20 wherein the endothelial cell is involved in the vascularisation of tumours.

22. A method according to any of claims 19-21 wherein the promoter sequence is selected from the following list: VEGFR-1; VEGFR-2; VEGFR-3; brain specific, endothelial glucose-1-transporter; endoglin; B61 receptor; endothelin B; mannose-6-phosphate; IL-1α; IL-1β; IL-1 receptor promoter.

23. A method according to any of claims 1-14 wherein the cell is a virally infected cell.

24. A method according to claim 23 wherein the virually infected cell is infected with; Human Immunodeficiency Virus; Human T Cell Leukamia Virus (1 & 2); Ebola virus; papilloma virus (eg); papovavirus; rhinovirus; poliovirus; herpesvirus; adenovirus; Epstein barr virus; influenza virus.

25. A method according to claim 24 wherein the HPV is selected from the following: HPV-2; HPV-6; HPV-11; HPV-16, HPV-18, HPV-31, HPV-33, HPV-52, HPV-54; HPV-56; HPV-5 and HPV-8.

26. A method according to claim 25 wherein the HPV is HPV-16.

27. A method according to any of claims 24-26 wherein the promoter is the interferon β.

28. A method according to any of claims 24-26 wherein the virally induced promoter is selected from EB-1; EB-2 or EB-3.
